(19) United States
(12) Patent Application Publication
(10) Pub. No.: US 2025/0201359 A1
(43) Pub. Date: Jun. 19, 2025

(54) METHOD FOR EVALUATING ATTACHMENT OF DEVELOPMENTALLY DISABLED CHILDREN AND PROVIDING PROMOTION THERAPY PROGRAM, AND SYSTEM THEREOF

(71) Applicant: eMOTIV CO., LTD., Seoul (KR)

(72) Inventor: Jung Sang MIN, Seoul (KR)

(21) Appl. No.: 18/948,360

(22) Filed: Nov. 14, 2024

(30) Foreign Application Priority Data
Dec. 15, 2023 (KR) 10-2023-0182878

(51) Int. Cl. G16H 10/20; G16H 15/00; G16H 20/70; G16H 50/30

(52) U.S. Cl. CPC G16H 10/20; G16H 15/00; G16H 20/70; G16H 50/30

(57) ABSTRACT

The present disclosure relates to a method of evaluating an attachment relationship between a developmentally disabled child and a caregiver, and providing a program for promoting the attachment relationship in response to a result of the evaluation, and a system of executing the same. One embodiment of the present disclosure may include an evaluation step of evaluating, by a server computer, a degree of a child's attachment and a degree of a caregiver's parenting sensitivity from answers submitted by a user using a terminal device, deriving the user's attachment category from a result of the evaluation, and selecting a promotion therapy program; and a promotion therapy program providing step of transmitting, by the server computer, a promotion therapy program to the user's terminal device, and allowing the user to execute the promotion therapy program on the terminal device.

### CROSS-REFERENCE TO RELATED APPLICATION

This application claims priority under 35 U.S.C § 119 to Korean Patent Application No. 10-2023-0182878 filed in the Korean Intellectual Property Office on Dec. 15, 2023, the entire contents of which are hereby incorporated by reference.

### FIELD OF THE INVENTION

The present disclosure relates to a method of evaluating an attachment relationship between a developmentally disabled child and a caregiver, and providing a program for promoting the attachment relationship in response to a result of the evaluation, and a system of executing the same.

### BACKGROUND OF THE RELATED ART

Developmental disorders are a condition in which development that should have occurred at a given age has not been achieved, and are characterized by delays and abnormalities in social relationships, communication, and cognitive development. In the case of developmental disorders, the growth rate in language, cognition, movement, and sociality is significantly slower than that of peers, so the self-help ability that can be utilized in real life may be reduced.

Developmental disorders can be classified into several types, including autism spectrum disorder (ASD), reactive attachment disorder (RAD), and attention deficit hyperactivity disorder (ADHD). Because a child with such a developmental disorder may have an attachment difficulty, forming and promoting an attachment relationship between a child and his or her caregiver may be an important issue.

In order to evaluate a degree of attachment in a child, a “strange situation procedure” (SSP), which evaluates a kid's reaction by exposing the kid to an unfamiliar space and observing the kid's reaction when separated from or reunited with his or her caregiver, and an “attachment Q-set” method, which scores a kid's behavior according to items describing various behaviors involving attachment, or the like may be used.

However, conventional evaluation methods have disadvantages of requiring professionally trained test personnel, being expensive, and requiring a space for a face-to-face test. Furthermore, because it is a test conducted based on children with normal development, there is also a problem in that it includes questions that are not suitable for evaluating a degree of attachment in a child with a developmental disorder.

In order to treat developmental disorders, an attachment promotion therapy program (APT), responsive teaching, parents-child interaction therapy, and the like may be used.

However, conventional treatment methods also have disadvantage of requiring professionally trained treatment personnel and incurring high costs.

### CITATION LIST

### Patent Literature

Korean Patent No. 10-2427137

### SUMMARY OF THE INVENTION

An aspect of the present disclosure is to solve the foregoing problems, and to provide a method of evaluating an attachment relationship between a developmentally disabled child and a caregiver, and providing a program for promoting the attachment relationship in response to a result of the evaluation, and a system of executing the same.

Furthermore, another aspect of the present disclosure is to provide a method evaluating an attachment relationship between a developmentally disabled child and a caregiver, using a mobile device and an application to reduce spatial and temporal constraints and increase accessibility, and a method of recommending treatment content in response to a result of the evaluation.

In order to achieve the foregoing objectives, one embodiment of the present disclosure may include an evaluation step of evaluating, by a server computer, a degree of the child's attachment and a degree of a caregiver's parenting sensitivity from answers submitted by a user using a terminal device, deriving the user's attachment category from a result of the evaluation, and selecting a promotion therapy program; and a promotion therapy program providing step of transmitting, by the server computer, a promotion therapy program to the user's terminal device, and allowing the user to execute the promotion therapy program on the terminal device.

In one embodiment, in the evaluation step, the server computer may assign a score to each answer submitted by the user in response to the evaluation items of the child's attachment test, and add up the assigned scores to calculate the child's attachment evaluation score.

In one embodiment, in the evaluation step, the server computer may assign a score to each answer submitted by the user in response to the evaluation items of the caregiver's parenting sensitivity test, and add up the assigned scores to calculate the caregiver's parenting sensitivity evaluation score.

In one embodiment, in the evaluation step, the server computer may combine the child's attachment level and the caregiver's parenting sensitivity level to classify the user's attachment category.

In one embodiment, the server computer may classify the user's attachment category as category A when both the child's attachment level and the caregiver's parenting sensitivity level are “high”, classify the user's attachment category as category B when one of the child's attachment level and the caregiver's parenting sensitivity level is “high” and the other one is “low”, and classify the user's attachment category as category C when both the child's attachment level and the caregiver's parenting sensitivity level are “low”.

In one embodiment, the server computer may select the type of a promotion therapy program to be provided to the user or select the level of the promotion therapy program in response to the classified user's attachment category.

In one embodiment, in the promotion therapy program providing step, the server computer may transmit at least one of a game in which the child and the caregiver cooperate to make one dish from among a pancake, a cookie, and an ice cream, content displaying a cooking recipe step by step, content capturing and decorating a photo with an image of a dish created from the game in which the user makes the dish, and content viewing a photo captured and decorated by the user to the user's terminal device.

In one embodiment, the method may further include a reporting step of processing, by the server computer, a result of evaluating a degree of the child's attachment and a degree of the caregiver's parenting sensitivity and a result of performing the user's promotion therapy program, and transmitting the processed results to the user's terminal device.

In one embodiment, in the reporting step, an attachment measurement report including at least one of the child's attachment evaluation score and attachment level, the caregiver's parenting sensitivity score and parenting sensitivity level, and the user's attachment category; and a program report including a result of performing a game in which the child and caregiver cooperate to make one dish from among a pancake, a cookie, and an ice cream may be processed and transmitted to the user's terminal device.

Another embodiment of the present disclosure may include a server computer having an evaluation module that evaluates a degree of a child's attachment and a degree of a caregiver's parenting sensitivity from answers submitted by a user using a terminal device, and derives the user's attachment category from a result of the evaluation to select a promotion therapy program; and a treatment module that transmits a promotion therapy program to the user's terminal device, receives a result of performing, by the user, the promotion therapy program on the terminal device, and stores the received result therein.

In another embodiment, the server computer may further include a reporting module that processes a result of evaluating a degree of the child's attachment and a degree of the caregiver's parenting sensitivity, and transmits the processed result to the user's terminal device.

The present disclosure may evaluate a degree of attachment of a child with a developmental disorder and a degree of parenting sensitivity of a caregiver, classify into a category, and then select a promotion therapy program corresponding to the category. The selected promotion therapy program may include performing relatively easy and difficult cooperative behaviors, help a child's emotional development through interactive experiences, and encourage natural interactions between the child and his or her caregiver.

Furthermore, a degree of attachment and a degree of parenting sensitivity, as well as a result of performing the promotion therapy program may be visualized and provided as a report, thereby allowing a user to easily understand a result of the evaluation and a result of the game execution.

Accordingly, the method of the present disclosure may be less expensive and more accessible than conventional evaluation and treatment methods, and may help strengthen a child's communication skills, empathy, and sense of accomplishment, and promote a sense of bond between the child and his or her caregiver.

### BRIEF DESCRIPTION OF THE DRAWINGS

FIG. 1 is a diagram briefly showing a system of evaluating an attachment of a developmentally disabled child and providing a promotion therapy program service according to one embodiment of the present disclosure.

FIG. 2 is a diagram briefly showing a configuration included in a server computer in one embodiment of the present disclosure.

FIG. 3 is a diagram showing a user interface displayed on a user terminal device in one embodiment of the present disclosure.

FIG. 4 is a diagram briefly showing a method of evaluating an attachment of a developmentally disabled child and providing a promotion therapy program service according to one embodiment of the present disclosure.

FIG. 5A is a diagram showing an evaluation process in one embodiment of the present disclosure. FIG. 5B is a diagram showing an example of evaluation items for a child's attachment test and a caregiver's parenting sensitivity test in one embodiment of the present disclosure.

FIGS. 6A to 6C are diagrams showing execution processes of first to third games, respectively, included in a promotion therapy program in one embodiment of the present disclosure.

FIGS. 7A to 7C are diagrams each showing an execution process of additional content included in a promotion therapy program according to one embodiment of the present disclosure.

FIGS. 8A and 8B are diagrams showing reports processed by a reporting module in one embodiment of the present disclosure.

FIG. 9 is a diagram showing a configuration of a computer device according to one embodiment of the present disclosure.

### DETAILED DESCRIPTION OF THE PREFERRED EMBODIMENT

The following description illustrates the principles of the present disclosure. It will thus be appreciated that those skilled in the art will be able to devise various devices that, although not explicitly described or shown in this specification, implement the principles of the present disclosure and are included in the concept and scope of the present disclosure. Furthermore, all conditional terms and embodiments recited herein are intended only for pedagogical purposes to aid the reader in understanding the principles of the invention, and are to be construed as being without limitation to such specifically recited embodiments and conditions.

Moreover, all detailed description herein reciting the principles, aspects, and embodiments of the invention as well as specific embodiments thereof are intended to encompass both structural and functional equivalents thereof. Additionally, it should be understood that such equivalents include both currently known equivalents as well as equivalents to be developed in the future, that is, any elements developed to perform the same function regardless of its structure.

Therefore, for example, it will be appreciated by those skilled in the art that the block diagrams presented herein represent the conceptual view of an exemplary circuit that embodies the principles of the invention. Similarly, it should be understood that any flow charts, flow diagrams, state transition diagrams, pseudocodes, and the like represent various processes which may be substantially represented in computer readable media and so executed by a computer or processor, whether or not such a computer or processor is explicitly shown.

The functions of various elements including a processor or a functional block represented as a concept similar thereto and shown in the accompanying drawings may be provided using hardware having a capability to execute appropriate software as well as dedicated hardware. When provided by the processor, the functions may be provided by a single dedicated processor, a single shared processor, or a plurality of individual processors, and some thereof may be shared with one another.

In addition, it should be understood that explicit use of the terms presented as processors, controls, or concepts similar thereto should not be interpreted by exclusively quoting hardware having an ability of executing software, and should be understood to implicitly include, without limitation, digital signal processor (DSP) hardware, and a ROM, a RAM and a non-volatile memory for storing software. Other known common hardware may also be included.

In the claims of this specification, an element expressed as a means for performing a function described in the detailed description is intended to include, for example, any method of performing a function including a combination of circuit elements or any form of software including firmware/microcode, and the like, which perform the function, and is combined with suitable circuitry for executing the software to perform the function. It should be understood that since functions provided by the various recited means are combined with one another and are combined with a scheme demanded by the claims in the present disclosure defined by the claims, any means capable of providing these functions are equivalent to means recognized from this specification.

The foregoing objects, features and advantages will be more obvious through the following detailed description associated with the accompanying drawings, and accordingly, the technological concept of the present disclosure can be easily implemented by a person having ordinary skill in the art to which the present disclosure pertains. In describing the present disclosure, moreover, the detailed description will be omitted when a specific description for publicly known technologies to which the present disclosure pertains is judged to obscure the gist of the present disclosure.

Hereinafter, preferred embodiments according to the present disclosure will be described in detail with reference to the accompanying drawings.

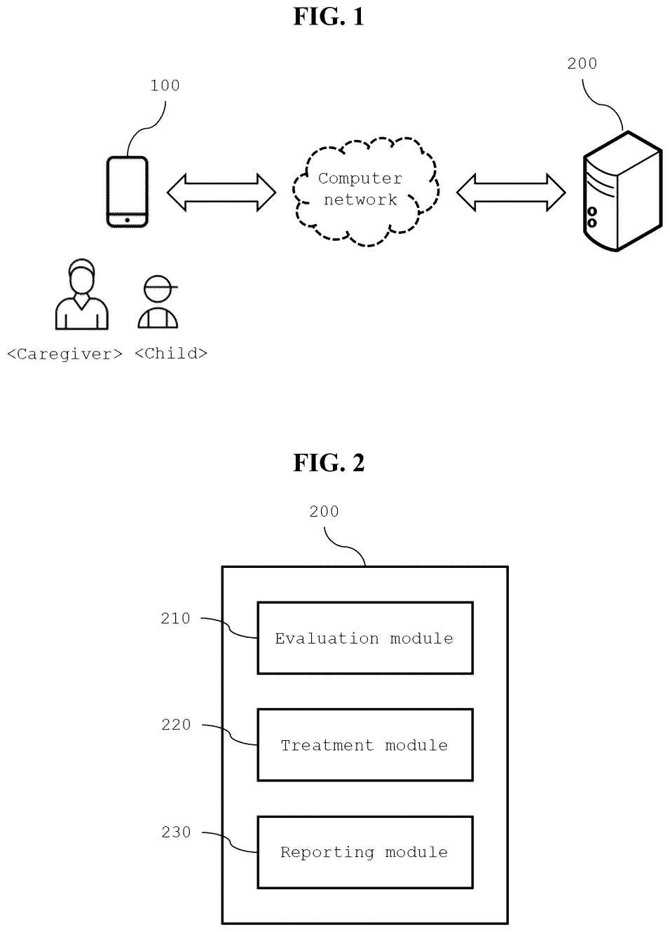

FIG. 1 is a diagram briefly showing a system of evaluating an attachment of a developmentally disabled child and providing a promotion therapy program service according to one embodiment of the present disclosure.

A system of evaluating an attachment of a developmentally disabled child and providing a promotion therapy program service (hereinafter referred to as a “system”) according to one embodiment of the present disclosure may each include a user terminal device 100 and a server computer 200.

The user terminal device 100 may be a computing device used by a child and a caregiver to diagnose or treat a developmental disorder. A child and a caregiver (collectively referred to as a “user”) may input answers corresponding to evaluation items on the user terminal device 100, interact with a promotion therapy program, and check a result of the evaluation and a result of performing the promotion therapy program.

The user terminal device 100 may be a portable mobile device such as a smart phone, a tablet computer, a laptop computer, a PDA, a smart watch, or a video game console. In one embodiment of the present disclosure, a case in which the user terminal device 100 is a mobile device is described as an example, but the user terminal device 100 is not limited to a mobile device and may also be, for example, a desktop computer, a nettop computer, a workstation, a set-top box, or the like, and may use the service of the present disclosure through an interaction with the server computer 200.

The server computer 200 may be a computing device that provides a child and his or her caregiver with evaluation items for diagnosing developmental disorders and a promotion therapy program corresponding to the responses, and calculates a result of the evaluation and a result of performing the promotion therapy program.

The server computer 200 may be a single computing device or a collection of a plurality of computing devices connected to one another via a computer network. The server computer 200 may include, for example, a plurality of rack-mounted servers, blade servers, and the like, and may be connected to a network device such as a router.

The user terminal device 100 and the server computer 200 may be connected via a computer network. The computer network may be, for example, the Internet, or one or more of the following subsets of the Internet: a personal area network (PAN), a local area network (LAN), a campus area network (CAN), a metropolitan area network (MAN), a wide area network (WAN), a radio access network (RAN), or an Internet area network (IAN). Each component may transmit or receive data using a data communication protocol such as TCP/IP.

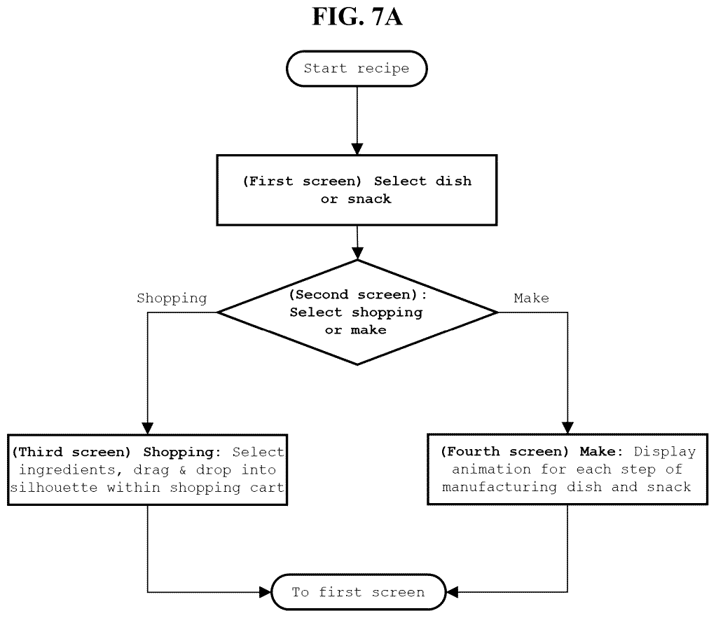

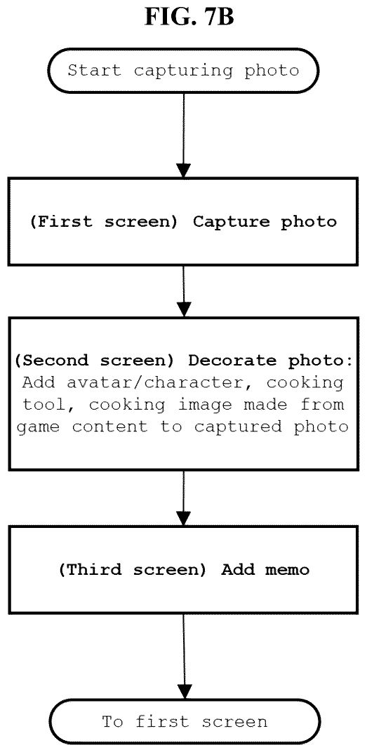

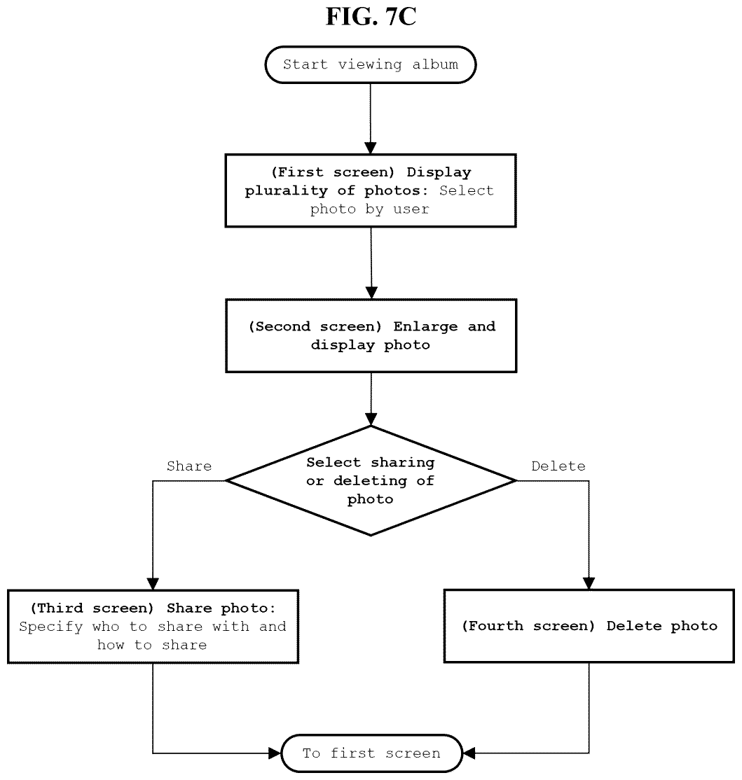

The configuration of a computing device operating as the user terminal device 100 and the server computer 200 will be described in detail in FIG. 7.

FIG. 2 is a diagram briefly showing a configuration included in a server computer in one embodiment of the present disclosure.

The server computer 200 may each include an evaluation module 210, a treatment module 220, and a reporting module 230.

The evaluation module 210 may evaluate a degree of a child's attachment and a degree of a caregiver's parenting sensitivity using a questionnaire survey or evaluation items. Furthermore, the evaluation module 210 may derive, in response to a user's response, the user's attachment category and select a promotion therapy program.

The treatment module 220 may provide a promotion therapy program to the user terminal device 100. The promotion therapy program is, for example, game software that can form an attachment relationship or strengthen a sense of bond between a child and a caregiver, and may be a game whose type or level is selected in response to responses to a questionnaire survey or evaluation items.

The reporting module 230 may process a result of evaluation for a questionnaire survey or evaluation items and a result of performing a promotion therapy program, and provide the processed results to the user terminal device 100.

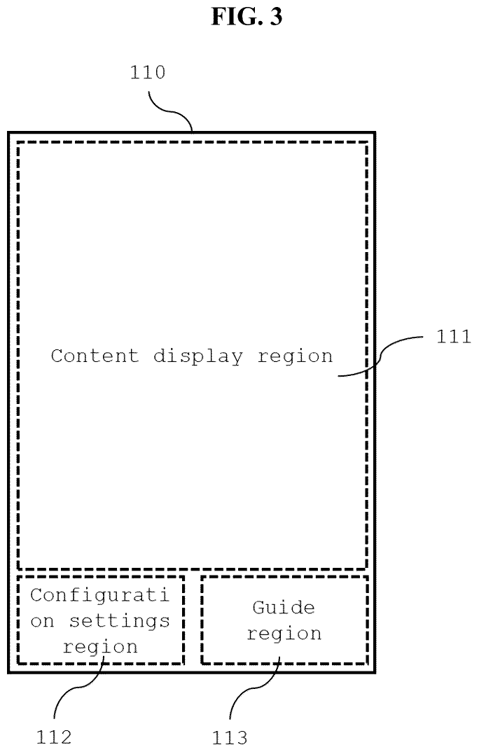

FIG. 3 is a diagram showing a user interface displayed on a user terminal device in one embodiment of the present disclosure.

Application software is installed on the user terminal device 100, and the user terminal device 100 may execute instructions included in the installed application software to display a user interface 110 as shown in FIG. 3. The application software may be a web browser, or one of a mobile, PC, or set-top box application programmed to interact with the server computer 200.

The application software may receive and display a user interface implemented as a web page from the server computer 200, or read and display a user interface programmed within the application software.

In the user interface 110, a content display region 111, a configuration settings region 112, and a guide region 113 may be displayed, and a user may make an input into each region.

The content display region 111 displays a questionnaire survey or evaluation items provided by the evaluation module 210, and the user may enter answers thereto in the content display region 111.

Furthermore, a promotion therapy program (e.g., game content) provided by the treatment module 220 is played and displayed in the content display region 111, and the user may operate the promotion therapy program in the content display region 111. The input and operation may be carried out through a touch screen located in a region where the content display region 111 is displayed on a user terminal device 100, or carried out by another input device (keyboard, mouse, etc.), and a result corresponding to the input and operation may be displayed in the content display region 111.

Furthermore, a result of evaluation for the questionnaire survey or evaluation items provided by the reporting module 230 and a result of performing the promotion therapy program may be displayed in the content display region 111.

In the configuration settings region 112, a menu may be displayed that allows the user to make detailed settings regarding content display and playback. For example, the appearance of the displayed or played content may be changed according to the user's taste and needs, such as by changing the quality, filter effects, and the like, of video and static image content, changing the font, size, color, and the like, of text in a document, changing the type of background music, effect music, guidance voice, and the like, or adjusting the volume of sound being played.

The configuration settings region 112 may be displayed in a reduced form or in the form of a button, and when the user selects the configuration settings region 112 (e.g., by touching the configuration settings region or moving a cursor and then performing a pressing operation), an enlarged form or a pop-up window may appear, and detailed settings for content display and playback may be made therein.

In the guide region 113, a guidance document that is helpful in using the user interface 110 or the service of the present disclosure may be displayed.

The guide region 113 may be displayed in a reduced form or in the form of a button, and when the user selects the guide region 113, an enlarged form or a pop-up window appears, in which guidance text may be displayed.

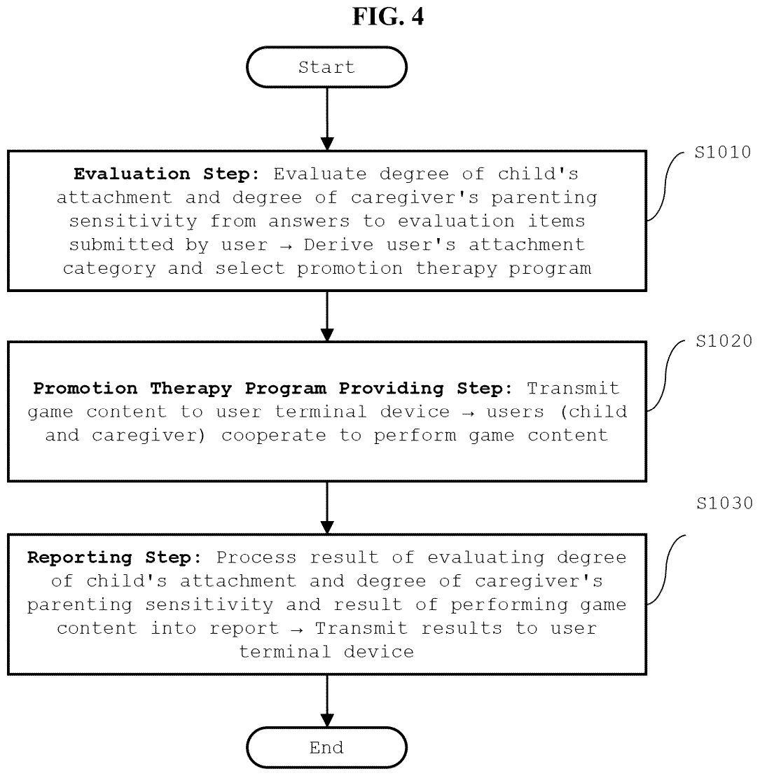

FIG. 4 is a diagram briefly showing a method of evaluating an attachment of a developmentally disabled child and providing a promotion therapy program service according to one embodiment of the present disclosure.

In an evaluation step S1010, the evaluation module 210 may evaluate a degree of a child's attachment and a degree of a caregiver's parenting sensitivity from answers submitted by a user to a questionnaire survey or evaluation items. Furthermore, the evaluation module 220 may derive the user's attachment category from a result of the evaluation and select a promotion therapy program.

In a promotion therapy program providing step S1020, the treatment module 220 may provide the promotion therapy program to the user terminal device 100.

The treatment module 220 may transmit game content to the user terminal device 100 as an example of the promotion therapy program selected by the evaluation module 210.

Users (a child and a caregiver) may perform game content cooperatively on the user terminal device 100, and thus an interaction between the child and the caregiver may occur, thereby promoting a sense of bond between the two. Furthermore, the treatment module 220 may receive and store a result of the user's execution within the game content.

In a reporting step S1030, the reporting module 230 may process a result of evaluating a degree of the child's attachment and a degree of the caregiver's parenting sensitivity and a result of performing the promotion therapy program, and provide the processed results to the user terminal device 100.

The details of each step will be described with reference to the following drawings.

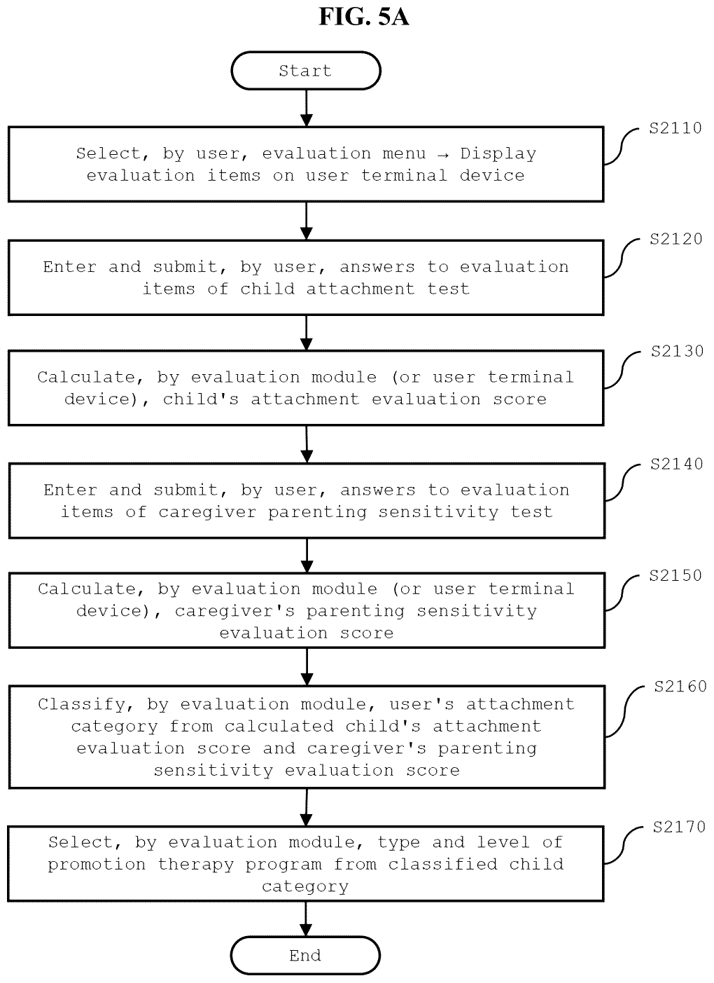

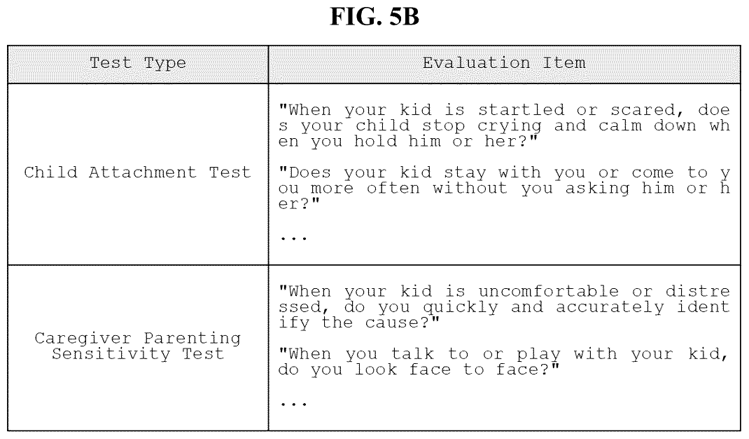

FIG. 5A is a diagram showing an evaluation process in one embodiment of the present disclosure. FIG. 5B is a diagram showing an example of evaluation items for a child's attachment test and a caregiver's parenting sensitivity test in one embodiment of the present disclosure.

When a user (child or caregiver) selects an evaluation menu in the user interface 110 displayed on the user terminal device 100, the evaluation module 210 may transmit a web page containing a questionnaire survey or evaluation items or transmit formatted data to the user terminal device 100. The formatted data may be data stored in a format defined by, for example, json, xml, yaml, or the like (step S2110)

The user terminal device 100 may parse the received web page and then render the parsed web page in the content display region 111, or process the formatted data into a document containing a questionnaire survey or evaluation items and display the processed document in the content display region 111.

The user may input answers to a plurality of evaluation items included in an attachment test, respectively, through a touch screen of the content display region 111 or an input device of the user terminal device 100 (step S2120).

The child's attachment test may include items evaluating a usual degree of the child's attachment as observed by the caregiver. For example, questions such as “When your kid is startled or scared, does your child stop crying and calm down when you hold him or her?” or “Does your kid stay with you or come to you more often without you asking him or her?” may be included.

Furthermore, multiple-choice options may be given for the questions, and scores may be assigned for respective options. For example, “Not at all”, “No”, “Average”, “Yes”, and “Very much” may be given as multiple-choice options, and scores may be sequentially assigned 1, 2, 3, 4, and 5.

When the user selects answers to evaluation items and then selects a submit menu, the user terminal device 100 may transmit the entered answers to the evaluation module 210. The evaluation module 210 may store the information of the received answers together with the user's (child's or caregiver's) identification information and the received visual information to manage the child's evaluation history. At this time, the evaluation module 210 may store the information as a file or build a database and add it as a record in a table.

The evaluation module 210 may add up the scores assigned in response to the received answers to calculate the child's attachment evaluation score. Alternatively, the user terminal device 100 may add up the scores assigned in response to the answers entered by the user, and then calculate the child's attachment evaluation score, and transmit the calculated score to the evaluation module 210 together with the entered answers. The evaluation module 210 may store the calculated evaluation score in a file or database (step S2130).

The user may input answers to a plurality of evaluation items included in a parenting sensitivity test, respectively, through a touch screen of the content display region 111 or an input device of the user terminal device 100 (step S2140).

A caregiver's parenting sensitivity test may include questions that evaluate a degree of the caregiver's parenting sensitivity to a child. For example, questions such as “When your kid is uncomfortable or distressed, do you quickly and accurately identify the cause?” and “When you talk to or play with your kid, do you look face to face?”

Furthermore, multiple-choice options may be given for the questions, and scores may be assigned for respective options. For example, “Not at all”, “No”, “Average”, “Yes”, and “Very much” may be given as multiple-choice options, and scores may be sequentially assigned 1, 2, 3, 4, and 5.

When the user selects answers to evaluation items and then selects a submit menu, the user terminal device 100 may transmit the entered answers to the evaluation module 210. The evaluation module 210 may store the information of the received answers together with the user's (child's or caregiver's) identification information and the received visual information to manage the child's evaluation history. At this time, the evaluation module 210 may store the information as a file or add it as a record in a table included in a database.

The evaluation module 210 may add up scores assigned in response to the received answers to calculate the caregiver's parenting sensitivity evaluation score. Alternatively, the user terminal device 100 may add up the scores assigned in response to the answers entered by the user, and then calculate the caregiver's parenting sensitivity score, and transmit the calculated score to the evaluation module 210 together with the entered answers. The evaluation module 210 may store the calculated evaluation score in a file or database (step S2150).

The evaluation module 210 may determine the child's attachment level and the caregiver's parenting sensitivity level from the calculated child's attachment evaluation score and the calculated caregiver's parenting sensitivity evaluation score. For example, when the child's attachment evaluation score is higher than a first reference score, the child's attachment level may be determined as “high”, and when it is lower than the first reference score, the child's attachment level may be determined as “low”. Furthermore, when the caregiver's parenting sensitivity score is higher than a second reference score, the caregiver's parenting sensitivity level may be determined as “high”, and when it is lower than the second reference score, the caregiver's parenting sensitivity level may be determined as “low”.

The evaluation module 210 may classify the user's attachment category by combining the determined child's attachment level with the caregiver's parenting sensitivity level. For example, when the child's attachment level and the caregiver's parenting sensitivity level are both “high”, it may be classified as category A. Furthermore, when one of the child's attachment level and the caregiver's parenting sensitivity level is “high” and the other one is “low”, it may be classified as category B. Furthermore, when the child's attachment level and the caregiver's parenting sensitivity level are both “low”, it may be classified as category C (step S2160).

The evaluation module 210 may select the type of a promotion therapy program or the level of a promotion therapy program to be performed based on the classified user's attachment category. For example, when the user's attachment category is classified as category A, the level of game content to be provided as a promotion therapy program may be selected as 3. Furthermore, when the user's attachment category is classified as category B, the level of the game content may be selected as 2, and when the user's attachment category is classified as category C, the level of the game content may be selected as 1 (step S2170).

The evaluation module 210 may process a web page or formatted data including the child's attachment evaluation score and attachment level, the caregiver's parenting sensitivity score and parenting sensitivity level, the child's category information, and the type and level of the selected promotion therapy program. The evaluation module 210 may transmit the processed web page or formatted data to the user terminal device 100. Furthermore, the user terminal device 100 may parse the received web page and then render the parsed web page in the content display region 111, or reprocess the formatted data and display the reprocessed data in the content display region 111.

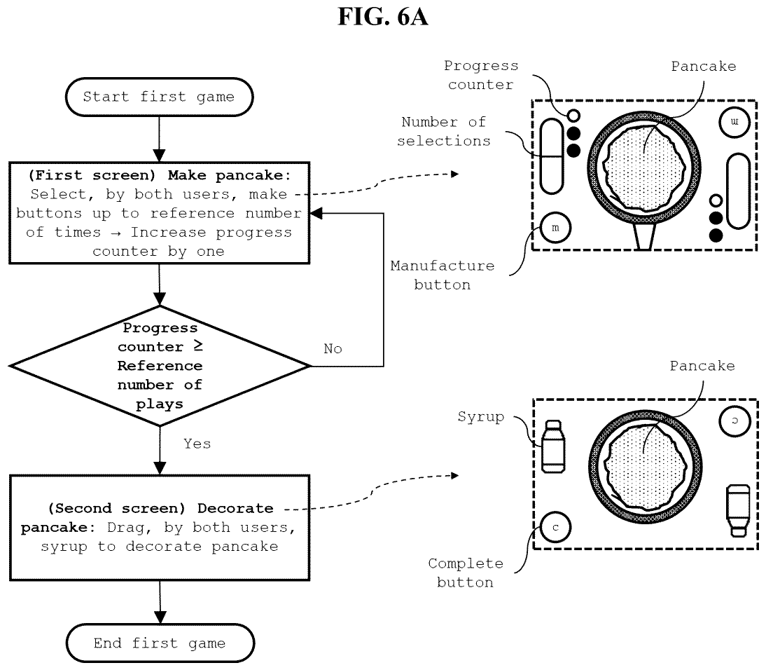

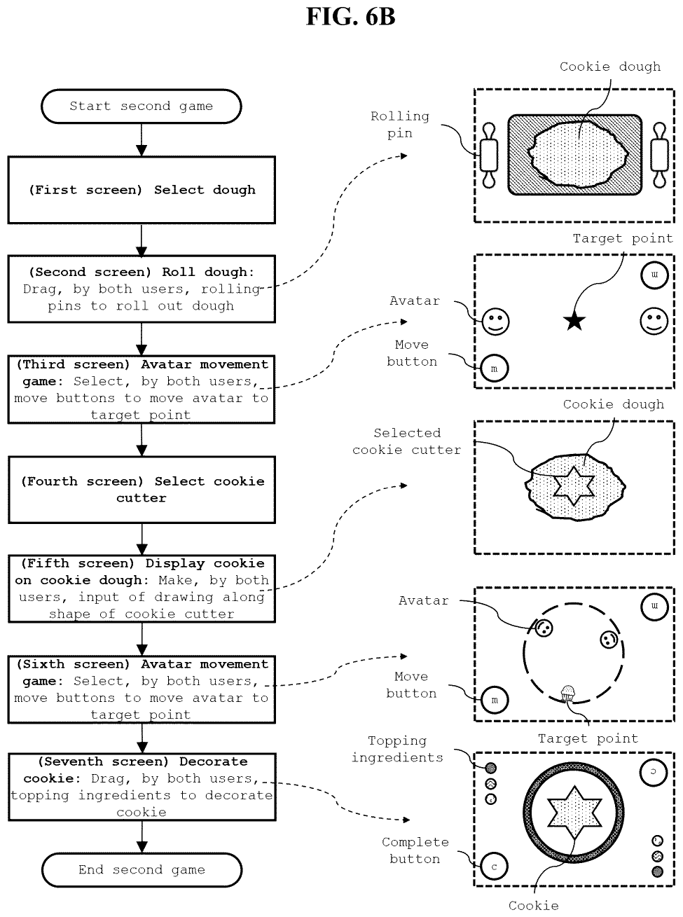

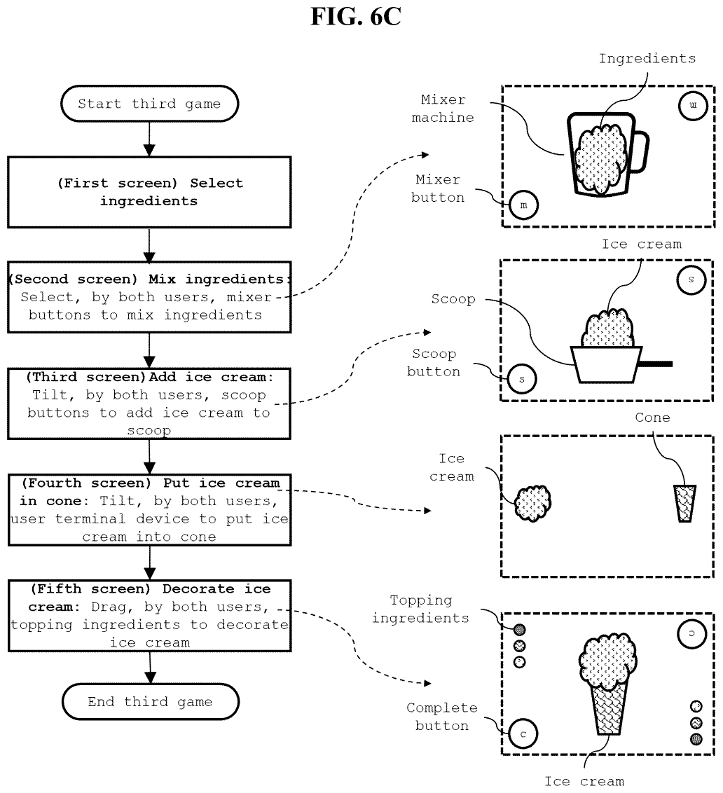

FIGS. 6A to 6C are diagrams showing execution processes of first to third games, respectively, included in a promotion therapy program in one embodiment of the present disclosure.

The treatment module 220 may transmit content to the user terminal device 100 in response to the type and level of the promotion therapy program selected by the evaluation module 210. The content provided to the user may be, for example, game software.

The user terminal device 100 may display a list of received content in the content display region 111. The user may select one content item from the content display region 111, and the user terminal device 100 may execute the content selected by the user. There may be a plurality of content items displayed in the content display region 111, and the user may select and execute one of them. In addition, the content display region 111 may display a level of content determined by the evaluation module 210, and the user may select the level to execute the content. The level may represent, for example, a degree of difficulty of game content (easy, normal, hard, etc.).

Prior to selecting content, the user may register photos of a children and a caregiver, and select avatars or animation characters to be displayed when executing the content.

As an example of content, a first game illustrated in FIG. 6A is a game in which a child and a caregiver cooperate to make a pancake. A child and a caregiver may execute the first game on one user terminal device 100. However, the present disclosure may not be limited thereto, and the child and the caregiver may also execute the first game on their own user terminal devices.

A first screen of the first game may display manufacture buttons at both ends thereof, respectively. Furthermore, an image that indicates a number of times the user has selected a manufacture button and a progress counter may be displayed thereon.

One user (child or caregiver) may select one manufacture button consecutively up to a reference number of sections (e.g. 10 times). For example, the user may take an operation of selecting a manufacture button by pressing a touch screen in a region corresponding to the manufacture button or by pressing an input device of the user terminal device 100.

Once one user has completed an operation of selecting a manufacture button consecutively up to a reference number of selections in his or her turn, the turn may be passed to the other user. The other user (caregiver or child) may select the other manufacture button consecutively in his or her turn up to a reference number of times.

When both users have completed an operation of selecting the manufacture button in their turns consecutively up to a reference number of selections, the progress counter may be increased by one. Furthermore, in the content display region 111, an image of a pancake may be displayed to correspond to the progress counter.

When the progress counter is below a reference number of plays (e.g. 3), both the users may be allowed to re-execute an operation of selecting the make button in their turns consecutively up to the reference number of selections. Otherwise, when the progress counter is above the reference number of plays, it may proceed to a next screen.

A second screen may display syrups and complete buttons at both ends thereof, respectively. Both the users may simultaneously or respectively select and drag syrups. At this time, as the syrup moves, an animation of contents coming out of the syrup may be displayed in the content display region 111. The user may decorate the image of the pancake by selecting and dragging the syrup.

Both the users may decorate the image of the pancake and then select the complete button to finish. The game may end when both the child and the caregiver select the complete button. When the game ends, an animation showing the pancake the user has created and decorated may be displayed in the content display region 111, and then a screen for selecting game content may be displayed again.

A second game illustrated in FIG. 6B is a game in which a child and his or her caregiver cooperate to make a cookie. The child and the caregiver may execute the second game on one user terminal device 100, or may respectively execute the second game on their own user terminal devices 100.

A first screen of the second game may allow the user to select a dough (e.g. a dough made of ingredients such as chocolate, strawberry, mint, etc.). Upon selecting a next button displayed on the screen, it may proceed to a next screen.

A second screen may display rolling pins at both ends thereof, respectively.

When one user (child or caregiver) drags one rolling pin to the top of the content display region 111 while selecting both ends thereof (e.g., pressing, by the user, a touch screen in a region where the rolling pin is displayed, or moving a cursor and then performing a pressing operation), an animation may be displayed in which the rolling pin moves, while wiggling, to the top of the content display region 111 and the dough grows larger.

When the other user (caregiver or child) drags the other rolling pin to the bottom of the content display region 111, an animation may be displayed in which the rolling pin moves, while wiggling, to the bottom of the content display region 111 and the dough grows larger.

When both the users have completed an operation of moving the rolling pin in their turns, the progress counter may be increased by one. When the progress counter is above the reference number of plays, it may proceed to a next screen.

A third screen may display move buttons, and avatars or characters selected by the users at both ends thereof, respectively. When both the users simultaneously or respectively select the move buttons in a consecutive manner, an animation may be displayed in which the avatars or characters move, while wiggling, toward a target point in the content display region 111.

If two avatars or characters move to the target point within a reference period of time, or if both the users select the button consecutively up to a reference number of selections, then a reward may be assigned to the avatars or characters. The reward may be represented by stars, coins, heart icons, and the like.

Furthermore, the user may repeatedly perform the game executed on the third screen, and when the number of plays exceeds a reference number of times (e.g., 5 times), it may proceed to a next screen.

A fourth screen may display a cookie cutter. The user may select a cookie cutter using the given reward. Depending on the reward the user has received, the types of cookie cutters available to select therefrom may also vary. When the user selects a cookie cutter, it may proceed to a next screen.

A fifth screen may display a cookie dough in the center thereof, and allows the shape of the cookie cutter selected by the user to be drawn on the cookie dough. Both the users may respectively start at a location in the shape of the cookie cutter drawn on the cookie dough, and make an input of drawing along the shape of the cookie cutter. Once both the users have drawn along the shape of the cookie cutter, it may proceed to a next screen.

A sixth screen may display move buttons at both ends thereof, respectively, and an avatar or character selected by the user may be displayed on a shape made up of several platforms in the center thereof. When the user selects a button, an animation of an avatar or character moving may be displayed in the content display region 111.

When both the users have completed an operation of selecting a button once in their turns, the progress counter may be increased by one. When the progress counter is above a reference number of selections, an animation of the avatar or character reaching a target point (cookie) is displayed in the content display region 111, and it may proceed to a next screen.

A seventh screen may display an image of a cookie created in the shape of a cookie cutter selected by the user. Furthermore, a plurality of topping ingredient images may be displayed. Both the users may respectively drag, while selecting, topping ingredients to decorate a cookie.

Both the users may decorate an image of the cookie and then select the complete button to finish. The game may end when both the child and the caregiver select the complete button. When the game ends, an animation showing the cookie the user has created and decorated may be displayed in the content display region 111, and then a screen for selecting game content may be displayed again.

A third game illustrated in FIG. 6C is a game in which a child and his or her caregiver cooperate to make an ice cream. The child and the caregiver may execute the third game on one user terminal device 100, or may respectively execute the third game on their own user terminal devices 100.

A first screen of the third game allows the user to select ingredients (e.g. chocolate, strawberry, mint, etc.). Upon selecting a next button displayed on the screen, it may proceed to a next screen.

A second screen may display mixer buttons at both ends thereof, respectively.

One user (child or caregiver) may each select the mixer button in his or her turn. Furthermore, both the users may select the mixer buttons simultaneously as instructed.

When a section where one user selects a button and a section where both the users select buttons simultaneously have been passed, the progress counter may be increased by one, and an animation in which the ingredient in the mixer changes in response to the progress counter may be displayed in the content display region 111. When the progress counter is above the reference number of plays, it may proceed to a next screen.

A third screen may display scoop buttons at both ends thereof, respectively.

When one user selects an activated scoop button, an animation may be displayed in which a scoop icon moves, while wiggling, toward the top of the content display region 111. When one user selects an activated scoop button above a reference number of selections, the scoop button may be deactivated and the other scoop button may be activated.

When the other user selects an activated scoop button, an animation may be displayed in which a scoop icon moves, while wiggling, toward the bottom of the content display region 111. When the other user selects an activated scoop button above a reference number of selections, the scoop button may be deactivated and the other scoop button may be activated, and the progress counter may be increased by one. Furthermore, in response to the progress counter, an animation of the ice cream being scooped up by a scoop growing larger may be displayed in the content display region 111, and when the progress counter is above a reference number of plays, it may proceed to a next screen.

A fourth screen may display an ice cream chunk at one end thereof, and a cone at an opposite end thereof. Furthermore, an animation in which an ice cream moves while rolling in one direction may be displayed in the content display region 111.

Both the users may control a direction of movement of the ice cream chunk by tilting the user terminal device 100 or selecting a button of moving a cursor on an input device. When the ice cream chunk touches the cone more than a reference number of contacts, it may proceed to a next screen.

A fifth screen may display an image of an ice cream. Furthermore, a plurality of topping ingredient images may be displayed. Both the users may respectively drag, while selecting, topping ingredients to decorate an ice cream.

Both the users may decorate an image of the ice cream and then select the complete button to finish. The game may end when both the child and the caregiver select the complete button. When the game ends, an animation showing the ice cream the user has created and decorated may be displayed in the content display region 111, and then a screen for selecting game content may be displayed again.

FIGS. 7A to 7C are diagrams each showing an execution process of additional content included in a promotion therapy program according to one embodiment of the present disclosure.

As an example of additional content, recipe content illustrated in FIG. 7A is an animation that displays a cooking recipe step by step.

A first screen of the recipe content may display images of various types of dishes and snacks. When the user selects one dish or snack image from among them, it may proceed to a next screen.

A second screen may display buttons that allow a child and a caregiver to select activities they need to do in order to cook a dish or snack together. For example, a “shopping” button and a “make” button may be displayed, and each button may be selected to proceed to the corresponding screen.

When the user selects the “shopping” button, a third screen may be displayed showing images of ingredients for dishes and snacks and an image of a shopping cart. On the screen, the shopping cart may display a silhouette image of the ingredients. When selecting an ingredient image, a pop-up window or message describing the ingredient may be displayed.

The user may select an ingredient image, drag it to the shopping cart image, and then drop it. When the ingredient image points to the same ingredient as the silhouette image at a location where the ingredient image is placed, an animation of the ingredient being added to the shopping cart may be displayed in the content display region 111. Otherwise, a message indicating that the ingredient does not match may be displayed.

When the user selects the “make” button, a fourth screen may be displayed that shows step-by-step instructions for manufacturing the dish or snack. An animation may be displayed for each step of manufacturing the dish or snack.

Subsequent to completing the steps on the third and fourth screens, the first screen may be displayed to execute again or finish the recipe content.

Photo capturing content illustrated in FIG. 7B is to allow a user to capture a photo with a cooking image created from game content.

On a first screen of the photo capturing content, the user may capture a photo. The user may determine whether to use the captured photo or recapture a photo. When selecting to recapture a photo, a capture screen may be displayed again to capture the photo. When selecting to use the captured photo, it may proceed to a next screen.

On a second screen, the user may decorate a photo. The user may perform an editing task on the captured photo, such as adding an avatar or character, an image of a cooking tool, or an image of a dish the user has created from game content. When the user selects a complete button displayed on the screen, it may proceed to a next screen.

On a third screen, the user may add a memo to the photo. The user may add or not add a memo according to his or her selection.

Subsequent to completing the step on the third screen, the first screen may be displayed to execute again or finish the photo capturing content.

Album view content illustrated in FIG. 7C is to allow to a user to view a decorated photo from the photo capturing content.

A first screen of the album view content may display a plurality of photos decorated by the user. The user may select one photo from among them, and proceed to a next screen.

On a second screen, the photo selected by the user may be enlarged and displayed. The user may select a share or delete menu for photos.

When the user selects a share menu, a third screen may be displayed to select who to share with and how to share. When the user specifies who to share a decorated photo with and how to share (e.g. e-mail, messenger, SNS, etc.), the decorated photo may be sent in response thereto.

When the user selects the delete menu, a fourth screen asking whether to delete the photo may be displayed. When the user selects to delete a photo, the selected photo may be deleted.

Subsequent to completing the steps on the third and fourth screens, the first screen may be displayed to execute again or finish the album view content.

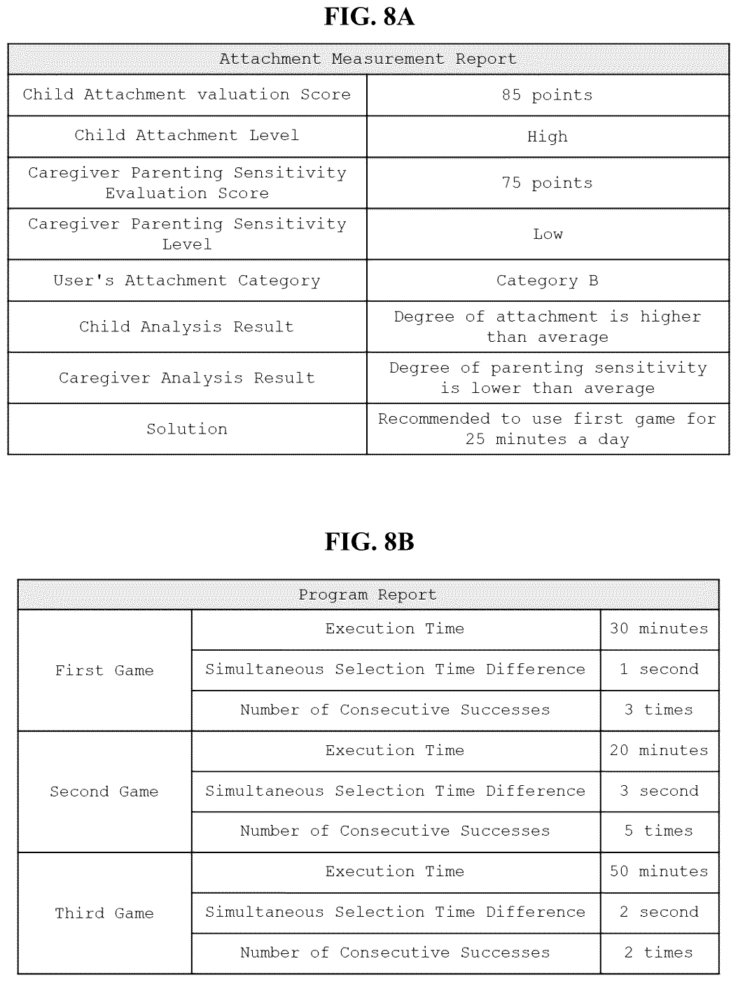

FIGS. 8A and 8B are diagrams showing reports processed by a reporting module in one embodiment of the present disclosure.

The reporting module 230 may process a result of evaluating, by the evaluation module 210, a degree of a child's attachment and a degree of a caregiver's parenting sensitivity to generate an attachment measurement report.

In a comprehensive analysis result of the attachment measurement report, a child's attachment evaluation score and attachment level, a caregiver's parenting sensitivity evaluation score and parenting sensitivity level, and a user's attachment category may each be shown as one or more figures, letters, or graphs.

A child analysis result may include information related to a child's attachment evaluation score and attachment level. A caregiver analysis result may include information related to a caregiver's parental sensitivity evaluation score and parental sensitivity level.

A solution may include a recommended activity, a communication method, and the like to promote an attachment relationship between a child and a caregiver.

The reporting module 230 may process a result of performing, by a user, a promotion therapy program to generate a program report.

The program report may include visualization of statistics on a result of performing first to third games. For example, statistics such as a period of time spent playing a game, a number of times a difference between the times both the users simultaneously touched a button was within a reference period of time, and a number of consecutive successful button selections when both users alternately selected the button may be included therein.

The program report may display an avatar or character selected by the user, and a distance between the avatars or characters may vary depending on a result of performing a game. For example, as a number of times a game is carried out increases, a distance between the avatars or characters selected by two users may be displayed as getting closer.

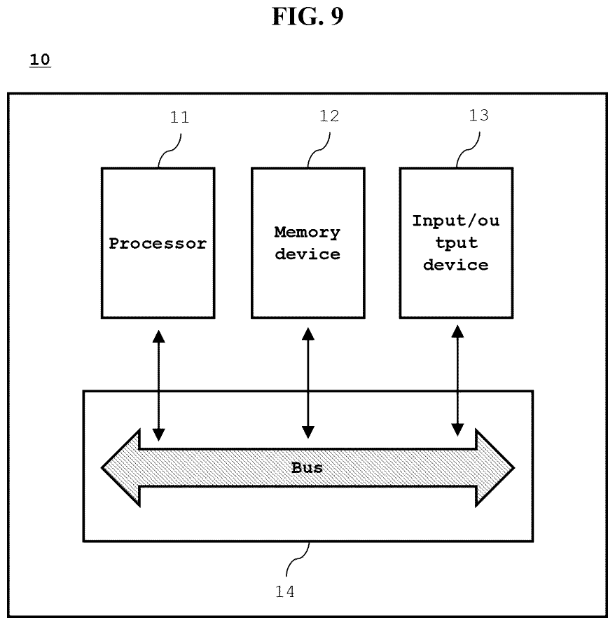

FIG. 9 is a diagram showing a configuration of a computer device according to one embodiment of the present disclosure.

The computing device 10 includes a processor 11, a memory device 12, an input/output device 13, and a system board 14.

The processor 11 executes an operation of reading, changing, or generating data used in one embodiment of the present disclosure. In addition, the processor 11 interprets and processes computer-readable instructions that execute the method of one embodiment of the present disclosure. The processor 11 may be a microprocessor including a control device that generates a control signal for interpreting and executing an instruction, an arithmetic and logic operation device that executes an arithmetic and logic operation instruction, a register that stores a plurality of instructions and a location of a next instruction to be executed, input/output data, a cache memory that temporarily stores data exchanged between the processor 11 and the memory device 12, and a system bus that is a path through which data moves within the processor 11.

The memory device 12 stores data processed or input/output within the computing device 10. Additionally, the memory device 12 stores computer-readable instructions that execute a method according to one embodiment of the present disclosure. The memory device 12 may include a main memory device and an auxiliary memory device. The main memory device may include a random access memory device or a flash memory device. The auxiliary storage device may include one or more of a hard disk drive, a solid state drive, a flash memory device, an optical disc drive, and a magnetic tape.

The input/output device 13 inputs data into the computing device 10 and outputs data to the outside. In addition, the input/output device 13 receives computer-readable instructions that execute a method according to one embodiment of the present disclosure. The input/output device may include an external input/output terminal and a driver device that processes the terminal. For example, the external input/output terminal may include one or more of a serial port, a parallel port, a small computer system interface (SCSI), a universal serial bus (USB), IEEE 1394, an external serial advanced technology attachment (e-SATA), and a Thunderbolt interface. In addition, the input/output device may include a network interface controller, and the network interface controller may be connected in a wired manner to a local area network (LAN) based on Ethernet, or connected in a wireless manner to a wireless local area network (WLAN) based on Wi-Fi.

The system board 14 connects between the processor 11, the memory device 12, and the input/output device 13, and provides a path for data processed by the computing device 10. The system board 14 may include an address bus, an instruction bus, a data bus, a chipset device that controls the buses, and a power system that supplies power to the components of the computer device.

The foregoing method according to the present disclosure may be produced as a program to be executed on a computer and stored in a computer-readable recording medium, and examples of computer-readable recording media include a ROM, a RAM, a CD-ROM, a magnetic tape, a floppy disk, an optical data storage device, and the like.

The computer-readable recording medium may be distributed over computer systems connected via a network, and stored and executed as computer-readable codes in a distributed manner. Furthermore, functional programs, codes, and code segments for implementing the method may be easily inferred by programmers in the technical field to which the present disclosure pertains.

In addition. while the preferred embodiments of the present disclosure have been shown and described above, it will be of course understood by those skilled in the art that various modifications may be made without departing from the gist of the disclosure as defined in the following claims, and it is to be noted that those modifications should not be understood individually from the technical concept and prospect of the present disclosure.

### DESCRIPTION OF SYMBOLS

100: User terminal device
110: User interface
111: Content display region
112: Configuration settings region
113: Guide region
200: Server computer
210: Evaluation module
220: Treatment module
230: Reporting module

What is claimed is:

1. A method of evaluating an attachment of a developmentally disabled child and providing a promotion therapy program service, the method comprising:
an evaluation step of evaluating, by a server computer, a degree of the child's attachment and a degree of a caregiver's parenting sensitivity from answers submitted by a user using a terminal device, deriving the user's attachment category from a result of the evaluation, and selecting a promotion therapy program; and
a promotion therapy program providing step of transmitting, by the server computer, a promotion therapy program to the user's terminal device, and allowing the user to execute the promotion therapy program on the terminal device.

2. The method of claim 1, wherein, in the evaluation step, the server computer assigns a score to each answer submitted by the user in response to the evaluation items of the child's attachment test, and adds up the assigned scores to calculate the child's attachment evaluation score.

3. The method of claim 1, wherein, in the evaluation step, the server computer assigns a score to each answer submitted by the user in response to the evaluation items of the caregiver's parenting sensitivity test, and adds up the assigned scores to calculate the caregiver's parenting sensitivity evaluation score.

4. The method of claim 1, wherein, in the evaluation step, the server computer combines the child's attachment level and the caregiver's parenting sensitivity level to classify the user's attachment category.

5. The method of claim 4, wherein the server computer classifies the user's attachment category as category A when both the child's attachment level and the caregiver's parenting sensitivity level are “high”, classifies the user's attachment category as category B when one of the child's attachment level and the caregiver's parenting sensitivity level is “high” and the other one is “low”, and classifies the user's attachment category as category C when both the child's attachment level and the caregiver's parenting sensitivity level are “low”.

6. The method of claim 4, wherein the server computer selects the type of a promotion therapy program to be provided to the user or selects the level of the promotion therapy program in response to the classified user's attachment category.

7. The method of claim 1, wherein, in the promotion therapy program providing step, the server computer transmits at least one of a game in which the child and the caregiver cooperate to make one dish from among a pancake, a cookie, and an ice cream, content displaying a cooking recipe step by step, content capturing and decorating a photo with an image of a dish created from the game in which the user makes the dish, and content viewing a photo captured and decorated by the user to the user's terminal device.

8. The method of claim 1, further comprising:
a reporting step of processing, by the server computer, a result of evaluating a degree of the child's attachment and a degree of the caregiver's parenting sensitivity and a result of performing the user's promotion therapy program, and transmitting the processed results to the user's terminal device.

9. The method of claim 8, wherein, in the reporting step, an attachment measurement report including at least one of the child's attachment evaluation score and attachment level, the caregiver's parenting sensitivity score and parenting sensitivity level, and the user's attachment category; and a program report including a result of performing a game in which the child and caregiver cooperate to make one dish from among a pancake, a cookie, and an ice cream are processed and transmitted to the user's terminal device.

10. A system of evaluating an attachment of a developmentally disabled child and providing a promotion therapy program service, the system comprising a server computer having:
an evaluation module that evaluates a degree of a child's attachment and a degree of a caregiver's parenting sensitivity from answers submitted by a user using a terminal device, and derives the user's attachment category from a result of the evaluation to select a promotion therapy program; and
a treatment module that transmits a promotion therapy program to the user's terminal device, receives a result of performing, by the user, the promotion therapy program on the terminal device, and stores the received result therein.

11. The system of claim 10, wherein the server computer further comprises:
a reporting module that processes a result of evaluating a degree of the child's attachment and a degree of the caregiver's parenting sensitivity, and transmits the processed result to the user's terminal device.
